(19) United States
(12) Patent Application Publication
(10) Pub. No.: US 2018/0249947 A1
(43) Pub. Date: Sep. 6, 2018

(54) CONSULTATION ADVICE USING ONGOING MONITORING

(71) Applicant: INTERNATIONAL BUSINESS MACHINES CORPORATION, Armonk, NY (US)

(72) Inventor: Dale Seegmiller Maudlin, Solana Beach, CA (US)

(21) Appl. No.: 15/449,306

(22) Filed: Mar. 3, 2017

(51) Int. Cl. A61B 5/00; A61B 5/01; A61B 5/021; A61B 5/08; A61B 5/145; A61B 3/12

(52) U.S. Cl. CPC A61B 5/486; A61B 5/01; A61B 5/021; A61B 5/0816; A61B 5/14532; A61B 5/14542; A61B 5/681; A61B 5/7246; A61B 5/7282; A61B 5/746; A61B 5/0024; A61B 3/1216; A61B 5/4806

(57) ABSTRACT

Consultation advice based on ongoing user monitoring is provided. In various embodiments, first physiological data of a user is collected by a wearable device for a first time period. From the first physiological data individualized physiological data statistics are determined. The individualized physiological data statistics are stored. Second physiological data of the user are collected by the wearable device for a second time period. The second physiological data are compared to the individualized physiological data statistics to detect an abnormality. The user is notified to seek medical attention.

### BACKGROUND

Embodiments of the present invention relate to medical monitoring, and more specifically, to providing consultation advice based on ongoing user monitoring.

### BRIEF SUMMARY

According to embodiments of the present disclosure, methods of and computer program products for medical monitoring are provided. First physiological data of a user is collected by a wearable device for a first time period. From the first physiological data individualized physiological data statistics are determined. The individualized physiological data statistics are stored. Second physiological data of the user are collected by the wearable device for a second time period. The second physiological data are compared to the individualized physiological data statistics to detect an abnormality. The user is notified to seek medical attention.

### BRIEF DESCRIPTION OF THE SEVERAL VIEWS OF THE DRAWINGS

FIG. 1 illustrates a system for consultation advice using ongoing monitoring according to embodiments of the present disclosure.

FIG. 2 illustrates a method for consultation advice using ongoing monitoring according to embodiments of the present disclosure

FIG. 3 depicts a computing node according to an embodiment of the present invention.

### DETAILED DESCRIPTION

Current patient monitoring technologies may be divided into those geared towards disease detection (e.g., EKG), and those geared towards fitness monitoring (e.g., Fitbit). Diagnostic devices such as an EKG are designed to be operated by a medical practitioner in a medical facility, and provide high detail but short duration data. In contrast, fitness oriented devices are designed to be used by a consumer and provide lower detail, but longer duration data.

In the case of fitness devices, health related metrics are only provided for user interpretation while disease diagnosis is left to specialized equipment. Accordingly, there remains a need for systems and methods that leverage the ongoing collection of data to provide early warnings of disease to a user.

In various embodiments, ongoing monitoring of subject physiological data is used to create an early warning signal in case of significant changes or early disease detection. In this way, a user may be prompted to contact a physician for follow up or diagnosis.

In various embodiments, a user profile is created that includes historical physiological data. Ongoing monitoring, for example through the use of existing fitness trackers, is performed. The ongoing data collection is used to update the user profile on an ongoing basis. Small changes in performance, for example, a change in stride or active heart rate may be indicative of an underlying pathology that is too subtle for the user to detect. In addition, the ongoing health data can be provided for other disease detection algorithms, for example those provided by Watson Healthcare technologies. Subtle changes can trigger a signal to a user to consult a physician without there being sufficient information for a full diagnosis. In this way, the data collected from ongoing monitoring can be leveraged to detect important variations that may not alone justify a diagnosis. Further variations of interest may be identified through data mining.

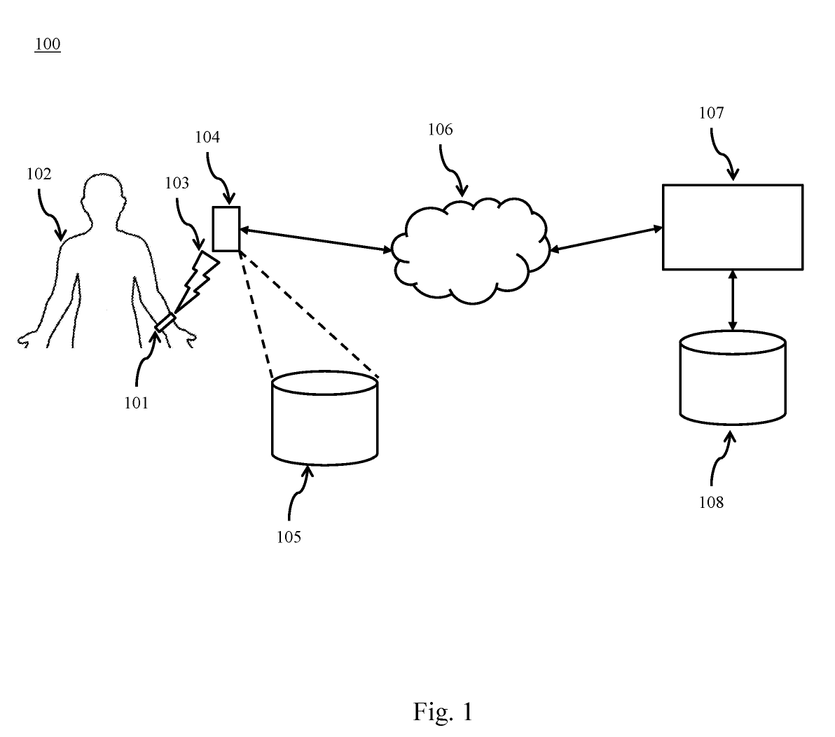

With reference now to FIG. 1, a system 100 for consultation advice using ongoing monitoring is illustrated according to embodiments of the present disclosure. One or more user monitoring device 101 is arrayed on one or more users 102. In some embodiments, monitoring device 101 is connected via a wireless connection 103 to a mobile device 104. For example, mobile device 104 may be a cellular phone. In some embodiments, monitoring device 101 is connected to mobile device 104 via personal area network such as Bluetooth. In some embodiments, monitoring device 101 communicates directly with network 106 without mobile device 104 as intermediary.

In some embodiments, as monitoring device 101 collects data regarding user 102, it is transmitted to mobile device 104 for storage in local data store 105. It will be appreciated that data may be synchronized according to various schedules, live, or at various intervals.

According to various embodiments, monitoring device 101 includes one or more biometric sensor. For example, the biometric sensors may include oximeters, heart rate sensors, blood pressure sensors, glucose sensors, pedometers, accelerometers (e.g., for measuring steps or falls), sensors for posture detection, temperature sensors, skin color sensors, eye color sensors, pulse sensors, or respiration sensors. Such sensors may be integrated into existing devices, such as a wearable watch or fitness tracker.

As data is collected regarding the user, an individual's baseline characteristics may be determined. For example, the heart rate and temperature of the individual on an average day may be charted. This longitudinal data allows detection of deviations from the norm. For example, a temperature that deviates more than 2 standard deviations from the mean may be indicative of illness. The baseline characteristics may include various statistical information such as mean, median, mode, standard deviation, etc.

Collecting data in this way allows systems according to the present disclosure to go beyond early diagnosis to perform a predictive or warning function. For example, variations in temperature, stride, gait, breathing pattern, sleep time, blood pressure, or pulse rate may be indicative of illness before a user is aware of symptoms. Moreover, biometric data as described herein is personalized, and thus provides a more reliable early warning of illness than comparing a user to the population average. For example, a given user may have an average temperature that differs from a relevant population average, and so detection of variation from the population mean is not necessarily abnormal.

In some embodiments, biometric data is transmitted via a wide area network 106 such as the internet to a remote server 107. Data may then be stored in data store 108. In some embodiments, data is anonymized at mobile device 104 before being sent to server 107. In various embodiments, server 107 may be a cloud server, may be located in a hospital or other medical facility, or may be located with a user's home or office. In some embodiments, the data is encrypted when placed in cloud storage in order to restrict access to only the subject user.

In various embodiments, server 107 performs analytics on aggregated user data. For example, latent associations between variables may be discovered through analysis of multiple users. In some embodiments, analysis is performed using various machine learning techniques, such as association rule learning algorithms and deep learning.

In some embodiments, once an biometric abnormality is detected, a user is notified to seek advice of a medical professional. If sufficient personalized information is not yet available, for example because the user is new, the user's biometrics may be compared to standard clinical guidelines instead. In various embodiments, notifications are provided via email, SMS, haptic feedback from a wearable device, an on-screen notification on a mobile device, an audio alerts, or any combination thereof. In some embodiments, a report may be provided to a medical professional summarizing the biometric data that led to the alert.

In some embodiments, integration is provided with electronic health record (HER) systems. For example, server 107 may provide biometric information or summary reports to existing HER systems via various interconnects known in the art such as HL7. In this way, data individualized data may be provided to various downstream, systems such as machine learning systems and computer-aided diagnosis systems.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

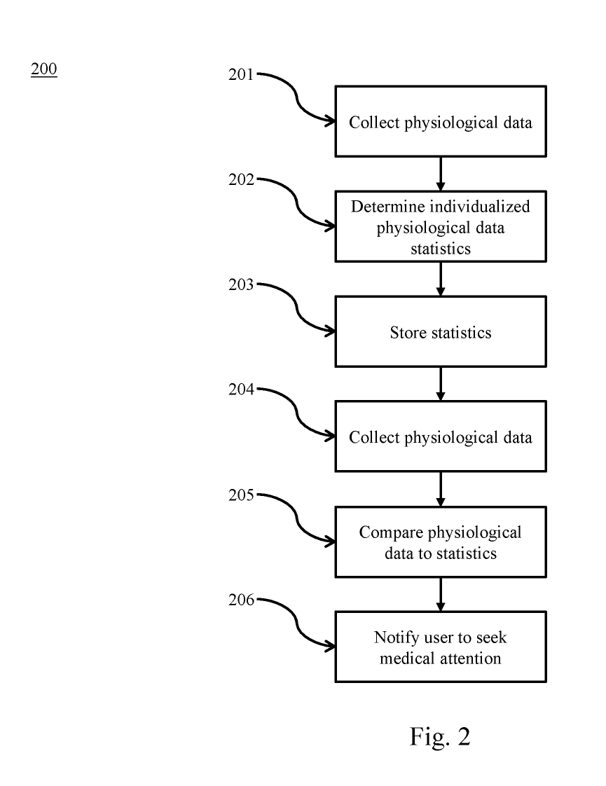

With reference now to FIG. 2, a method 200 for consultation advice using ongoing monitoring is illustrated according to embodiments of the present disclosure. At 201, first physiological data of a user is collected by a wearable device for a first time period. At 202, from the first physiological data individualized physiological data statistics are determined. At 203, the individualized physiological data statistics are stored. At 204, second physiological data of the user are collected by the wearable device for a second time period. At 205, the second physiological data are compared to the individualized physiological data statistics to detect an abnormality. At 206, the user is notified to seek medical attention.

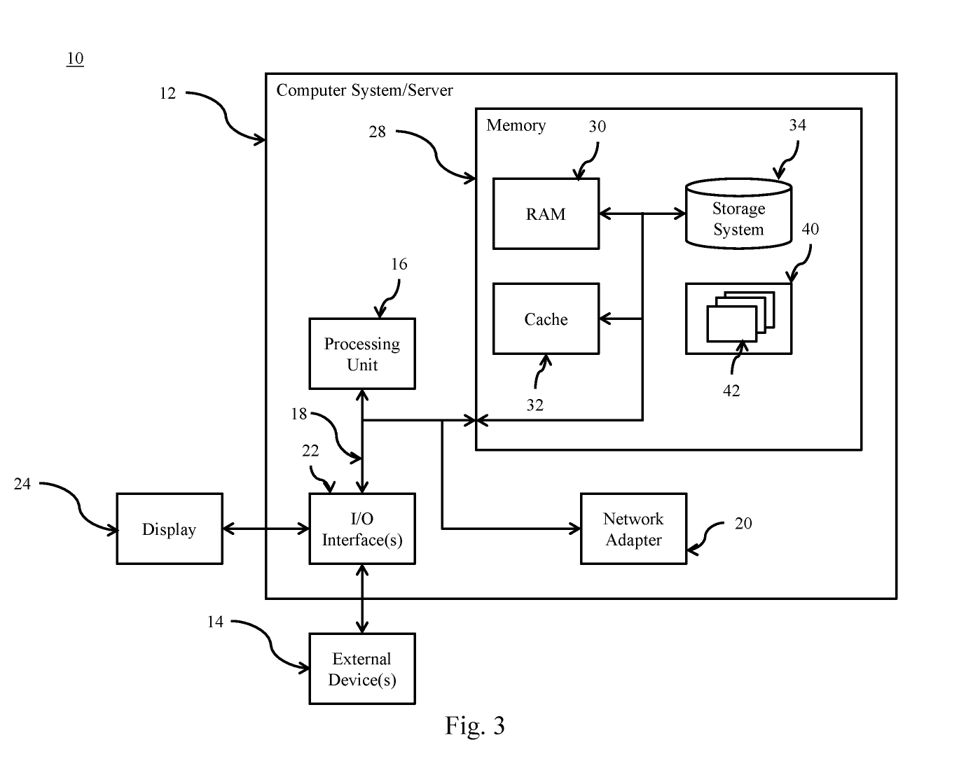

Referring now to FIG. 3, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 3, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
collecting first physiological data of a user by a wearable device for a first time period;
determining from the first physiological data individualized physiological data statistics;
storing the individualized physiological data statistics;
collecting second physiological data of the user by the wearable device for a second time period;
comparing the second physiological data to the individualized physiological data statistics to detect an abnormality;
notifying the user to seek medical attention.

2. The method of claim 1, wherein the physiological data comprise measurements of temperature, stride, gait, breathing pattern, sleep time, blood pressure, pulse rate, oxygen saturation, heart rate, blood pressure, glucose, steps, falls, posture, skin color, eye color, pulse, or respiration.

3. The method of claim 1, wherein the wearable device comprises a sensor.

4. The method of claim 3, wherein the sensor comprises an oximeter, heart rate sensor, blood pressure sensor, glucose sensor, pedometer, accelerometer, sensor for posture detection, temperature sensor, skin color sensor, eye color sensor, pulse sensor, or respiration sensor.

5. The method of claim 1, wherein the individualized physiological data statistics comprise a median and a standard deviation.

6. The method of claim 1, wherein notifying the user comprises sending an email or SMS.

7. The method of claim 1, further comprising:
generating a report comprising the individualized physiological data statistics and the second physiological data.

8. The method of claim 1, further comprising:
sending the second physiological data to an EHR system.

9. The method of claim 1, wherein collecting the first and second physiological data comprises:
sending the first and second physiological data from the wearable device to a second device via a personal area network.

10. A system comprising:
a wearable device;
a computing node comprising a computer readable storage medium having program instructions embodied therewith, the program instructions executable by a processor of the computing node to cause the processor to perform a method comprising:
collecting first physiological data of a user by the wearable device for a first time period;
determining from the first physiological data individualized physiological data statistics;
storing the individualized physiological data statistics;
collecting second physiological data of the user by the wearable device for a second time period;
comparing the second physiological data to the individualized physiological data statistics to detect an abnormality;
notifying the user to seek medical attention.

11. The system of claim 10, wherein the physiological data comprise measurements of temperature, stride, gait, breathing pattern, sleep time, blood pressure, pulse rate, oxygen saturation, heart rate, blood pressure, glucose, steps, falls, posture, skin color, eye color, pulse, or respiration.

12. The system of claim 10, wherein the wearable device comprises a sensor.

13. The system of claim 12, wherein the sensor comprises an oximeter, heart rate sensor, blood pressure sensor, glucose sensor, pedometer, accelerometer, sensor for posture detection, temperature sensor, skin color sensor, eye color sensor, pulse sensor, or respiration sensor.

14. The system of claim 10, wherein the individualized physiological data statistics comprise a median and a standard deviation.

15. The system of claim 10, wherein notifying the user comprises sending an email or SMS.

16. A computer program product for medical monitoring, the computer program product comprising a computer readable storage medium having program instructions embodied therewith, the program instructions executable by a processor to cause the processor to perform a method comprising:
collecting first physiological data of a user by a wearable device for a first time period;
determining from the first physiological data individualized physiological data statistics;
storing the individualized physiological data statistics;
collecting second physiological data of the user by the wearable device for a second time period;
comparing the second physiological data to the individualized physiological data statistics to detect an abnormality;
notifying the user to seek medical attention.

17. The computer program product of claim 16, wherein the physiological data comprise measurements of temperature, stride, gait, breathing pattern, sleep time, blood pressure, pulse rate, oxygen saturation, heart rate, blood pressure, glucose, steps, falls, posture, skin color, eye color, pulse, or respiration.

18. The computer program product of claim 16, wherein the wearable device comprises a sensor.

19. The computer program product of claim 18, wherein the sensor comprises an oximeter, heart rate sensor, blood pressure sensor, glucose sensor, pedometer, accelerometer, sensor for posture detection, temperature sensor, skin color sensor, eye color sensor, pulse sensor, or respiration sensor.

20. The computer program product of claim 16, wherein the individualized physiological data statistics comprise a median and a standard deviation.
